(19) United States
(12) Patent Application Publication
(10) Pub. No.: US 2020/0214689 A1
(43) Pub. Date: Jul. 9, 2020

(54) Hybrid Suture Anchor

(71) Applicant: CONMED CORPORATION, Utica, NY (US)

(72) Inventors: Adrian Bosworth, Bradenton, FL (US); Ryo Isshiki, Saint Petersburg, FL (US)

(73) Assignee: CONMED CORPORATION, Utica, NY (US)

(21) Appl. No.: 16/637,546

(22) PCT Filed: Aug. 9, 2018
(86) PCT No.: PCT/US18/45957

§ 371 Date: Feb. 7, 2020

Related U.S. Application Data
(60) Provisional application No. 62/543,516, filed on Aug. 10, 2017.

(51) Int. Cl. A61B 17/04

(52) U.S. Cl. CPC A61B 17/0401; A61B 2017/00898

(57) ABSTRACT

An anchor includes an expandable member configured to increase in size from a first pre-deployed condition to a second deployed condition; and a filament having a first filament end and a second filament end, and positioned in contacting relation to the expandable member in the second deployed condition. The anchor may also include a flat fibrous construct having a first end and a second end, and wherein the filament passes through the fibrous construct. The flat fibrous construct includes a first state in which the flat fibrous construct is uncompressed and extends along the longitudinal axis of the filament when in an unfolded and pre-deployed condition; and a second state in which the flat fibrous construct is compressed and expanded in a direction perpendicular to longitudinal axis of the filament in a deployed condition.

### CROSS-REFERENCE TO RELATED APPLICATION

The present application claims priority to and the benefit of U.S. Provisional Patent Application No. 62/543,516 filed on Aug. 10, 2017.

### BACKGROUND OF THE INVENTION

### 1. Field of the Invention

The present invention relates to an all-suture anchor construct for fixation in a bone hole or tunnel and, more particularly, to an all-suture anchor construct having an expandable portion with a length of a suture or filament positioned in contacting relation therewith upon deployment, and which may also include a flat section of woven suture tape, braid or fibrous construct with the length of suture woven or positioned therethrough.

### 2. Description of Related Art

Many orthopedic surgical and medical procedures require the fixation of one body to another body. Such bodies may include bone, soft tissue, and prosthetics. One body can be fixed in a position relative to another using connector devices, such as screws and suture anchors (e.g., cannulated knotless suture anchors and soft all-suture anchors). For example, various orthopedic surgeries (such as the reattachment of soft tissue to bone) require the insertion and fixation of a suture anchor within a bone hole (e.g., at a desired tissue reattachment location). Suture anchors can include “hard” suture anchors, and “soft” all-suture anchors.

As described in U.S. Pat. No. 8,409,252, for example, “non-soft,” “hard” or “rigid” suture anchors generally include a “hard” anchor body portion (that may or may not include inner and outer members) and a suture/filament portion. The anchor body of such suture anchors may be formed of a biocompatible and/or bioabsorbable material. These materials may be of such composition that they are reabsorbed by the body, e.g., during the healing process of the bone. Exemplary materials that are suitable for use in the inner and outer members include, but are not limited to, polyetheretherketone (“PEEK”), polylactic acid/beta-tricalcium phosphate (“PLA/Beta-TCP”) composites, ultra-high molecular weight polyethylene (“UHMWPE”), as well as other metallic, non-metallic, and polymeric materials.

Since soft anchors are commonly made entirely of suture materials, they are sometimes called “all-suture” anchors, and generally include a fibrous construct anchor body portion (or fibrous, braided or woven fabric-type structure such as a flexible web, as described in U.S. Pat. No. 9,173,652) and a suture or filament portion. Another example of a “soft” all-suture anchor is the Y-Knot® device. See, e.g., U.S. Pat. No. 9,826,971. Such soft all-suture anchors are often preferred by some orthopedic surgeons over the hard suture anchors because of their relative softness and usually excellent pull-outs strength among other reasons which should be understood by a person of ordinary skill in the art. In a traditional Y-Knot device, a suture filament is pierced entirely through a braid material a number of times, such that the suture passes through a “front” surface and a “back” surface.

There are at least two general, conventional methods for inserting a suture anchor within a bone. In one method, a bone hole is created and prepared using a drill bit. The drill bit is typically advanced through a drill guide to create the bone hole and then, a suture anchor is passed through or down the drill guide with an anchor inserter/installation device into the bone hole for deployment.

In a second method, the drilling step is eliminated in an attempt to avoid the aforementioned misalignment issue. A self-punching suture anchor, such as the Y-Knot® RC suture anchor, for example, is designed with an inserter that allows the anchor in the inserter to be directly positioned on the bone at the desired location. When the anchor in the inserter is positioned at the desired location, the inserter can be hammered, forcing the anchor directly into the bone.

Conventional methods and devices for inserting/deploying such all-suture anchors are known, examples of which are disclosed in U.S. Pat. No. 9,173,652.

In order to deploy correctly, conventional all-suture type anchors must expand diametrically and thus rely on the compressive failure of the sub-cortical cancellous bone at the implantation site. If however, the subcortical bone is extremely hard and dense, such as is found in the load bearing region of the acetabular rim of the hip joint, compressive failure of the bone may not occur, thus resulting in poor pull-out strength of the anchor.

Therefore, there is a need for a soft all-suture anchor construct that can be structured and composed of materials sufficient to greatly increase anchor pull-out strength in hard bone.

Description of the Related Art Section Disclaimer: To the extent that specific patents/publications/applications/products are discussed above in this Description of the Related Art Section or elsewhere in this disclosure, these discussions should not be taken as an admission that the discussed patents/publications/products are prior art for patent law purposes. For example, some or all of the discussed patents/publications/products may not be sufficiently early in time, may not reflect subject matter developed early enough in time and/or may not be sufficiently enabling so as to amount to prior art for patent law purposes. To the extent that specific patents/publications/applications/products are discussed above in this Description of the Related Art Section and/or throughout the application, the descriptions/disclosures of which are all hereby incorporated by reference into this document in their respective entirety(ies).

### BRIEF SUMMARY OF THE INVENTION

Embodiments of the present invention recognize that there are potential problems and/or disadvantages with conventional soft all-suture anchors (as discussed herein and above). Various embodiments of the present invention may be advantageous in that they may solve or reduce one or more of the potential problems and/or disadvantages discussed herein.

The present disclosure is directed to an inventive configuration, structure, and resulting function of a soft all-suture anchor that utilizes a hybrid combination of soft implantable materials. A hybrid soft all-suture anchor of an embodiment includes superior pull-out strength properties as compared to conventional soft all suture anchors. Embodiments of the present invention provide a better soft all-suture anchor for use in hard bone, due in part to a hybrid expanding component portion. These embodiments are also suitable for use in soft cancellous bone where there is a very thin or weak cortical layer.

In one embodiment, an all-suture anchor is disclosed and can include, but is not limited to, an expandable member/portion configured to increase in size from a first pre-deployed condition to a second deployed condition; and a filament having a first filament end and a second filament end, and positioned in contacting relation to the expandable member in the second deployed condition. The anchor may also include a flat fibrous construct having a first end and a second end, and wherein the filament passes through the fibrous construct. The flat fibrous construct includes a first state in which the flat fibrous construct is uncompressed and extends along the longitudinal axis of the filament when in an unfolded and pre-deployed condition; and a second state in which the flat fibrous construct is compressed and expanded in a direction perpendicular to longitudinal axis of the filament in a deployed condition. The structure, configuration, and functionality of the expandable member and of the fibrous construct (when part of an embodiment) help to set and hold the anchor in the bone hole in a post-deployment condition.

According to another embodiment, the all-suture anchor briefly described above in conjunction with an installation device is provided. The installation device can include, but is not limited to, a handle and a distal deployment end, which can be fork-shaped or other appropriate shape to sufficiently hold during deployment and to deploy the all-suture anchor within a bone hole.

According to yet another embodiment, a method of deploying the all-suture anchor briefly described above in a preformed bone hole (already drilled) can include, but is not limited to, the steps of: (i) providing the all-suture anchor briefly described above; and (ii) using the installation device to deploy the all-suture anchor into the preformed bone hole (preferably into cancellous bone below the cortex) by tensioning the free ends of the filament (applying a force on the free ends of the filament in a direction away from the bone hole). In brief, the tensile force applied to the suture tails causes the flat tape/fibrous construct to “form a clump” and “ball-up” underneath the cortical layer and thus provide fixation for the anchor. In an embodiment that includes an expandable portion (with or without the fibrous construct), an activator can be applied to cause expansion of the expandable member to deploy the anchor.

Suture material, sutures, or filaments as the terms are used and described herein, can include monofilament or multi-filament suture as well as any other metallic or non-metallic filamentary or wire-like material suitable for performing the function of a suture. This material can include both bioabsorbable and non-absorbable materials, and can be round, flat, or braided.

### BRIEF DESCRIPTION OF THE SEVERAL VIEWS OF THE DRAWING(S)

The present invention will be more fully understood and appreciated by reading the following Detailed Description in conjunction with the accompanying drawings. The accompanying drawings illustrate only typical embodiments of the disclosed subject matter and are therefore not to be considered limiting of its scope, for the disclosed subject matter may admit to other equally effective embodiments. Reference is now made briefly to the accompanying drawings, in which:

FIG. 1 is a perspective view digital photograph of a soft all-suture anchor in an unloaded (not loaded onto an installation device), pre-deployment configuration according to an embodiment;

FIG. 2A is a side view schematic representation of an embodiment of the all-suture anchor of FIG. 1 connected to an installation device in a pre-deployment configuration according to an embodiment;

FIG. 2B is a side view schematic representation of an embodiment of the all-suture anchor of FIG. 1 in a post-deployment configuration positioned in a bone hole according to an embodiment;

FIG. 2C is a side view schematic representation of portion of an alternative embodiment of the all-suture anchor according to an embodiment; and

FIG. 3 a side view digital photograph of an embodiment of the all-suture anchor of FIG. 1 in a post-deployment configuration after addition of an activator according to an embodiment.

### DETAILED DESCRIPTION OF THE INVENTION

in accordance with an embodiment of the present invention, the hybrid all-suture anchor can be formed from an expanding portion (as described below and illustrated by the accompanying figures), and any filament/suture and/or fibrous construct (as should be understood by a person of skill in the art in conjunction with a review of this disclosure). For example, the all-suture anchor as shown and described in U.S. Pat. No. 9,826,971, including the filament and the fibrous construct and installation device, can form part of embodiments of the present invention. In addition, the all-suture anchor shown and described in U.S. patent application Ser. No. 16/033,616, including the filament and the fibrous construct and installation device, can form part of embodiments of the present invention. An expanding portion, which can include any sponge-like material including, but not limited to, a cellulose fiber sponge material (or other biocompatible material), can be combined with any all-suture anchor (including, but not limited to, examples discussed herein and in U.S. Pat. No. 9,826,971 and U.S. patent application Ser. No. 16/033,616 as should be understood by those of ordinary skill in the art in conjunction with a review of this disclosure) to form an embodiment of the hybrid all-suture anchor of the present invention. In an alternative embodiment, the fibrous construct can be eliminated and the filament and expandable portion can act as a hybrid all-suture anchor.

Set forth below are example descriptions related to the structure and functionality of, and to a method associated therewith, a hybrid all-suture anchor of an embodiment of the present invention. Advantages of the invention are illustrated by the example descriptions set forth herein. However, the particular conditions and details are to be interpreted to apply broadly in the art and should not be construed to unduly restrict or limit embodiments of the invention in any way.

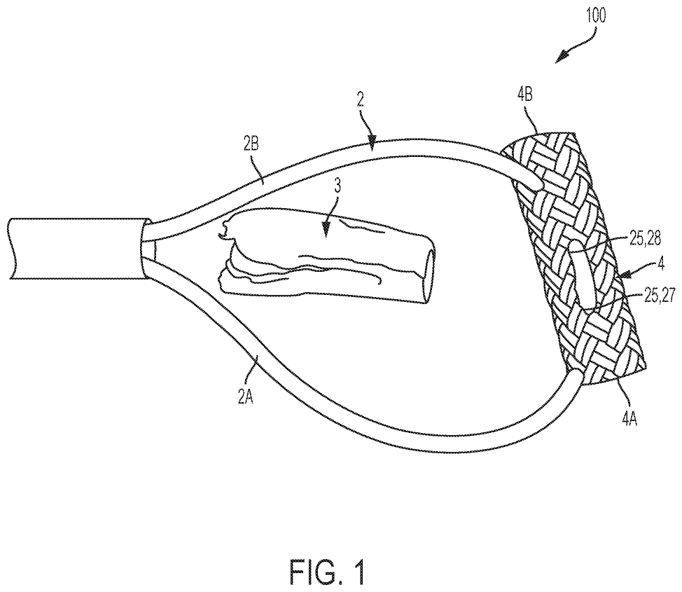

Referring now to the drawings, wherein like reference numerals refer to like parts throughout, there is seen in FIG. 1 a perspective view of a hybrid soft all-suture anchor 100 in a pre-deployment configuration, according to an embodiment. The hybrid all-suture anchor 100 can include, but is not limited to, a flat fibrous construct 4 having a first end 4A, a second end 4B. A filament 2 is shown having a first end 2A and a second end 2B, and woven, threaded, or otherwise passing through the fibrous construct 4 at passing locations 25, 27 and 25, 28. See U.S. Pat. No. 9,826,971 for a further description of the structural aspects of the filament and fibrous construct, which is part of this example of the invention (as should be understood by a person of ordinary skill in the art in conjunction with a review of this disclosure).

Still referring to FIG. 1, the hybrid soft all-suture anchor 100 also includes an expandable portion 3 in a non-expanded configuration and positioned proximately to the fibrous construct 4 and not attached to the filament 2. The expandable portion 3 can be positioned distally to the fibrous construct 4 in an alternative embodiment, or conjoined with the fibrous construct in another embodiment. The expandable portion 3 can be attached to the filament in the pre-deployment configuration in an alternative embodiment.

In an embodiment, the filament 2 is free to slide through the fibrous construct 4 (and the expandable portion 3 when attached thereto) such that the filament 2 can be removed from the fibrous construct 4 from the first end 4A of the fibrous construct 4 and/or the second end 4B of the fibrous construct 4. In accordance with an alternative embodiment, the filament is locked and not slidable through the fibrous construct 4 and/or the expandable portion 3 (when attached to the expandable portion 3).

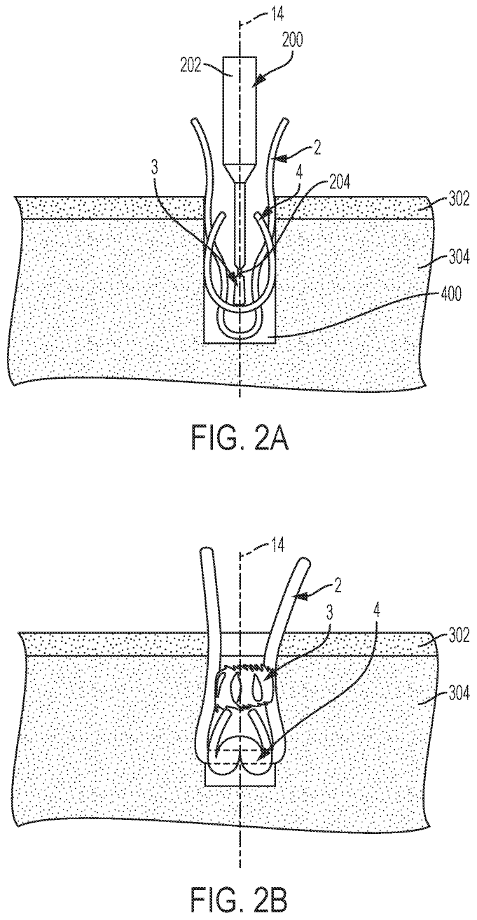

Turning now to FIGS. 2A and 2B, there are shown side view schematic representations of an embodiment of the all-suture anchor 100 in the pre-deployment and post-deployment configurations. As described above, the all-suture anchor 100 contains at least two sections: at least one suture 2 with a first end 2A and a second end 2B; and an anchor body/fibrous construct 4 with a first end 4A and a second end 4B, which is configured to form a portion of the anchor 100 that can increase in width, thickness and/or diameter and shrink in length as part of deployment. The all-suture anchor 100 also includes an expandable portion 3 which is configured to form a portion of the anchor 100 that can increase in size in the post-deployment configuration in response to an activator (as should be understood by a person of ordinary skill in the art in conjunction with a review of this disclosure).

As shown in FIG. 2A, the installation device in the pre-deployment configuration is provided. The all-suture anchor 100 is shown connected to the distal deployment end 204 of an installation device 200, which also includes a handle 202. The distal deployment end 204 and the all-suture anchor 100 are shown positioned in a bone hole 400 in cancellous bone 304 under the bone cortex 302. In order to deploy the all-suture anchor 100 (which can be connected to other tissue that needs to be brought into apposition to the bone, as should be understood by a person of ordinary skill in the art in conjunction with a review of this disclosure), the first end 2A and/or the second end 2B are pulled/tensioned in a direction away from the bone hole 400. The first end 2A and the second end 2B can be pulled/tensioned in a direction away from the bone hole 400 with or without the installation device 200 in place in the bone hole 400 (if installation device 200 is in place in the bone hole 400, it can act as a counter force to the tension force out of the hole 400 to assist with the deployment of the all-suture anchor 100). In addition, an activator can be added to the anchor to cause the expandable portion to expand to a second size greater than the first pre-deployment size. In one embodiment, the activator is water.

As shown in FIG. 2B, the anchor body/fibrous construct 4 is shown “shortened” and “expanded” in the post-deployment configuration and locked in the bone hole 400, which can be additive to the increase due to pleats formed by the fibrous construct 4 (which may also be part of the fibrous construct 4). The all-suture anchor 100, and, in particular, the fibrous construct 4 takes advantage of Poisson's ratio, which captures the following cause/effect relationship: compressing a material in a first direction causes the material to expand in direction perpendicular to the first direction (i.e., if compressed in the x-direction, the material will expand in the y-direction and/or z-direction), and stretching/lengthening a material in a first direction causes the material to contract in directions perpendicular to the first direction. Although, it is the anchor body/fibrous construct 4 that increases in width, thickness and/or diameter at deployment, it should be understood that the suture 2 can also play a role in the deployment of the anchor 100 even though the suture 2 may remain free to slide in some embodiments, and non-slidable in others (at least at a particular position or point in use) in relation to the anchor body 4. The suture 2 helps to position, align and support the anchor body 4 (as should be understood by a person of skill in the art in conjunction with a review of this disclosure).

In other words, the anchor body/fibrous construct 4 has two primary functions. First, it becomes a base for the suture 2 to slide within (within the column/lumen 6). Second, when compressed and/or pleated during deployment, the anchor body 4 becomes more compact in one direction thereby expanding outwardly and increasing its overall width, thickness or diameter to create a retention capacity. This action of having the anchor body 4 change in shape to increase its overall width, thickness or diameter is a useful characteristic which may be used advantageously to secure the anchor 100 in a hole 400 or against a bony or soft tissue. It is this combination of the expanding anchor body 4 coupled with the suture 2 remaining slidable (in some embodiments; and non-slidable in others, at least at a particular position or point in use) in relation to the anchor body 204 that render embodiments of the present invention ideal for the reattachment of soft tissue to bone or soft tissue to soft tissue where it is desirable to pass sliding knots to secure a repair.

Still referring to FIG. 2B, the expandable portion 3 is shown in the expanded second size, greater than the first smaller pre-deployment size, after exposure to the activator. The expandable portion expands greatly in volume when exposed to the activator, causing it to wedge in the bone hole 400 and lock the anchor 100 in place. In accordance with an embodiment, in order to tension the filament 2 to reattach soft tissue (not shown), the filament 2 can freely slide backward and forward through the fibrous construct 4 and through the expandable portion 3 (as may be necessary when connected to the expandable portion 3). In certain situations without the presence of fibrous construct 4, the free sliding filament 2 could potentially cut through the expandable portion 3 resulting in a less than optimum deployment of the all-suture anchor 100. As such, in some embodiments of the all-suture anchor 100 with or without the fibrous construct 4, a second short length of suture 2-1 could be wrapped or looped around the filament 2 (see FIG. 2C) to prevent sawing/cutting through the expandable portion 3 by the filament 2 when in contacting relation with the expandable portion 3.

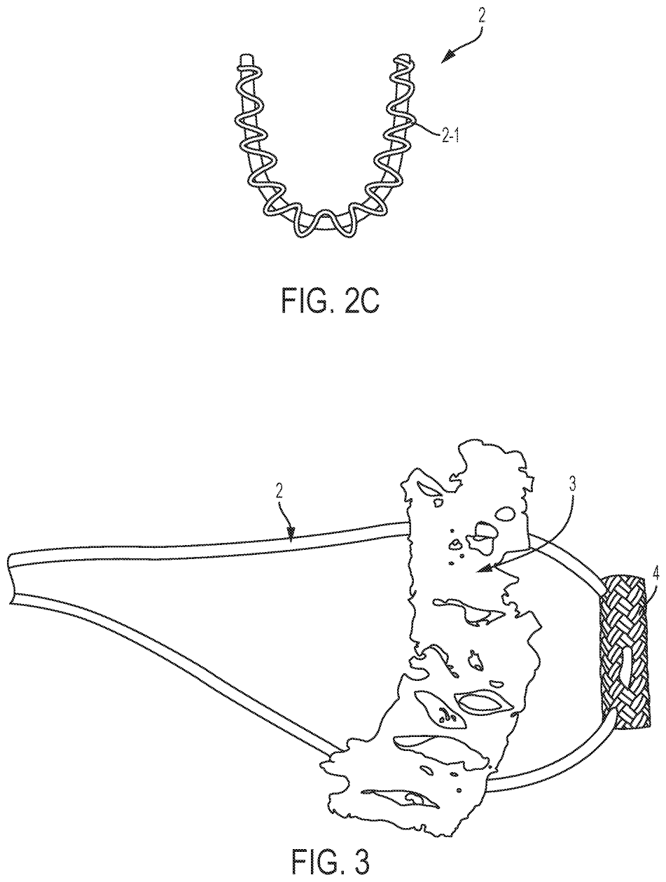

Turning to FIG. 3, a side view digital photograph of an embodiment of the all-suture anchor of FIG. 1 in a post-deployment configuration after addition of an activator according to an embodiment is shown. As shown, the expandable portion 3 has increased in size to a second deployed structural condition (bone hole is not shown to illustrate the extent of expansion of expandable portion 3), and the filament 2 is positioned through and/or in otherwise contacting relation with the expandable portion 3.

Similarly with respect to the filament 2 and fibrous construct 4 described above and the embodiments shown in FIGS. 2A-C, the expandable portion 3 can be a part of the all-suture anchor shown and described in U.S. patent application Ser. No. 16/033,616. The same structure and functionality of the expandable portion 3 described above and shown in FIGS. 2A-C applies to the embodiments of the all-suture anchor (with and without the fibrous construct) shown and described in U.S. patent application Ser. No. 16/033,616.

While embodiments of the present invention has been particularly shown and described with reference to certain exemplary embodiments, it will be understood by one skilled in the art that various changes in detail may be effected therein without departing from the spirit and scope of the invention as defined by claims that can be supported by the written description and drawings. Further, where exemplary embodiments are described with reference to a certain number of elements it will be understood that the exemplary embodiments can be practiced utilizing either less than or more than the certain number of elements.

What is claimed is:

1. An anchor for placement in or against tissue, the anchor comprising:
an expandable member configured to increase in size from a first pre-deployed condition to a second deployed condition; and
a filament having a first filament end and a second filament end, and positioned in contacting relation to the expandable member in the second deployed condition.

2. The anchor of claim 1, wherein the expandable member is configured to increase in size from the first pre-deployed condition to a second deployed condition upon the application if an activator.

3. The anchor of claim 2, wherein the activator is water.

4. The anchor of claim 1, further comprising a flat fibrous construct having a first fibrous construct end and a second fibrous construct end, and wherein the filament passes through the fibrous construct.

5. The anchor of claim 4, wherein the filament passes through the fibrous construct in two locations including a first passing location nearest a first end of the fibrous construct, a second passing location nearest a second end of the fibrous construct, the filament remaining free to slide through the first passing location and the second passing location such that the filament can be removed from the flat fibrous construct from the first end of the fibrous construct and the second end of the fibrous construct.

6. The anchor of claim 5, wherein the flat fibrous construct further comprises:
a first state in which the flat fibrous construct is uncompressed and extends along a longitudinal axis of the filament when in an unfolded and pre-deployed condition; and
a second state in which the flat fibrous construct is compressed and expanded in a direction perpendicular to the longitudinal axis of the filament in a deployed condition.

7. The anchor of claim 4, wherein the fibrous construct further comprises an open elongated column extending from a first elongated column end to a second elongated column end, and wherein the filament passes through and is positioned at least partially in the open elongated column.

8. The anchor of claim 7, wherein the flat fibrous construct further comprises:
a first state in which the flat fibrous construct is uncompressed and extends along a longitudinal axis of the filament when in an unfolded and pre-deployed condition; and
a second state in which the flat fibrous construct is compressed and expanded in a direction perpendicular to the longitudinal axis of the filament in a deployed condition.

9. The anchor of claim 7, wherein the filament is free to slide through the open column such that the filament can be removed from the open column from the first fibrous construct end and the second fibrous construct end.

10. The anchor of claim 7, wherein at least the first filament end and the second filament end extend outside of and beyond a respective elongated column end.

11. The anchor of claim 7, wherein the open elongated column is woven along an axis that is parallel to or along a central axis of the flat fibrous construct.

12. An anchor deployment system comprising:
an anchor including:
an expandable member configured to increase in size from a first pre-deployed condition to a second deployed condition; and
a filament having a first filament end and a second filament end, and positioned in contacting relation to the expandable member in the second deployed condition; and
an installation device including a distal deployment end on which the anchor is positioned and is configured to deploy the anchor into a bone hole.

13. The anchor placement system of claim 12, the distal deployment end of the installation device comprises a fork around which the anchor is positioned.

14. The anchor placement system of claim 12, wherein the expandable member is configured to increase in size from the first pre-deployed condition to a second deployed condition upon the application if an activator.

15. The anchor placement system of claim 12, further comprising a flat fibrous construct having a first fibrous construct end and a second fibrous construct end, and wherein the filament passes through the fibrous construct.

16. The anchor placement system of claim 15, wherein the filament passes through the fibrous construct in two locations including a first passing location nearest a first end of the fibrous construct, a second passing location nearest a second end of the fibrous construct, the filament remaining free to slide through the first passing location and the second passing location such that the filament can be removed from the flat fibrous construct from the first end of the fibrous construct and the second end of the fibrous construct.

17. The anchor placement system of claim 16, wherein the flat fibrous construct further comprises:
a first state in which the flat fibrous construct is uncompressed and extends along a longitudinal axis of the filament when in an unfolded and pre-deployed condition; and
a second state in which the flat fibrous construct is compressed and expanded in a direction perpendicular to the longitudinal axis of the filament in a deployed condition.

18. The anchor placement system of claim 15, wherein the fibrous construct further comprises an open elongated column extending from a first elongated column end to a second elongated column end, and wherein the filament passes through and is positioned at least partially in the open elongated column.

19. The anchor placement system of claim 18, wherein the flat fibrous construct further comprises:
a first state in which the flat fibrous construct is uncompressed and extends along a longitudinal axis of the filament when in an unfolded and pre-deployed condition; and
a second state in which the flat fibrous construct is compressed and expanded in a direction perpendicular to the longitudinal axis of the filament in a deployed condition.

20. The anchor of claim 7, wherein the filament is free to slide through the open column such that the filament can be removed from the open column from the first fibrous construct end and the second fibrous construct end.

21. A method of anchoring tissue to bone, comprising the steps of:
providing an anchor including:
an expandable member configured to increase in size from a first pre-deployed condition to a second deployed condition; and
a filament having a first filament end and a second filament end, and positioned in contacting relation to the expandable member in the second deployed condition; and
deploying the anchor into a bone hole.

22. The method of claim 21, wherein the step of deploying includes the step of tensioning at least one filament end in a direction away from the bone hole.

23. The method of claim 21, wherein the step of deploying includes the step of adding an activator to the expandable member to increase the size of the expandable member from the first pre-deployed condition to a second deployed condition.

24. The method of claim 22, wherein the anchor further comprises a flat fibrous construct having a first fibrous construct end and a second fibrous construct end, and wherein the filament passes through the fibrous construct.

25. The method of claim 24, wherein the step of tensioning further comprises the step of:
converting the flat fibrous construct from a first state in which the flat fibrous construct is uncompressed and extends along a longitudinal axis of the filament when in an unfolded and pre-deployed condition to a second state in which the flat fibrous construct is compressed and expanded in a direction perpendicular to the longitudinal axis of the filament in a deployed condition.
